(19) United States
(12) Patent Application Publication
(10) Pub. No.: US 2016/0067277 A1
(43) Pub. Date: Mar. 10, 2016

(54) USE OF XENON FOR LIMITING TUMOR CELL MIGRATION

(71) Applicant: L'Air Liquide, Societe Anonyme pour I'Etude et I'Eploitation des Procedes Georges Claude, Paris (FR)

(72) Inventors: Chui Fung CHONG, Paris (FR); Jan Pype, Herne (BE)

(21) Appl. No.: 14/783,351

(22) PCT Filed: Mar. 5, 2014
(86) PCT No.: PCT/FR2014/050488

§ 371 Date: Oct. 8, 2015

(30) Foreign Application Priority Data
Apr. 15, 2013 (FR) 1353394

(51) Int. Cl. A61K 33/00; A61K 45/06; A61K 9/00

(52) U.S. Cl. CPC A61K 33/00; A61K 9/007; A61K 45/06

(57) ABSTRACT

The invention relates to a gaseous composition containing xenon as active ingredient for use in preventing or limiting tumour cell migration and/or limiting or preventing metastasis formation in an individual suffering from cancer. The prevention or limitation of tumour cell migration is obtained through the action of the xenon on free cancer cells. The xenon acts on the free cancer cells carried by the blood. The effective volume proportion of xenon is between 5% and 79%, it is preferably at least 50%. The gaseous composition also contains a volume proportion of oxygen (O₂) of at least 21%. It is administered by inhalation or insufflation.

### CROSS-REFERENCE TO RELATED APPLICATIONS

This application is a 371 of International PCT Application

PCT/FR2014/050488 filed Mar. 5, 2014 which claims priority to French Patent Application No. 1353394 filed Apr. 15, 2013, the entire contents of which are incorporated herein by reference.

### BACKGROUND

The invention relates to the use of a gas composition containing gaseous xenon for preventing or for limiting tumor cell migration and/or thus limiting or preventing metastasis formation in a cancer patient, in particular for killing some or all of the cancer cells carried by the blood circulation in said patient.

Cancer is a disease that is characterized by an abnormally high level of cellular proliferation within a normal tissue or organ of the organism, threatening the latter's survival. Cancer cells are all derived from one and the same initial cell, namely the cancer initiating cell, which has acquired certain characteristics allowing it to divide indefinitely.

In the course of development of the disease, certain cells are released or become detached from the cancerous tissue or organ, and may migrate from their place of production to other places in the body. These free, migrating cells therefore give rise to other cancerous sites in the body, called metastases.

These migrating cells are released either spontaneously, or unintentionally during surgery performed on the patient.

In fact, one of the most widely used surgical techniques for eliminating cancers is based on removal of the cancerous site by surgery.

For this purpose, using a scalpel, laser beam or other device, the cancerous tissues, as well as a portion of the tissues not yet infected on which said cancerous tissues developed, are cut away. In this way, all of the cancerous tumor is removed, but also healthy tissues so as not to risk cutting directly into the tumor and thus spreading a large number of cancer cells, which might end up in the body fluids, especially blood and lymph.

Now, although this technique of cutting away the cancerous tissues is radical, in that it makes it possible to completely remove the cancerous tissues forming the cancerous site, it has the drawback that despite everything, it leads to release of migrating cancer cells, which may then be transported elsewhere in the body by being carried by the blood and may form metastases elsewhere in the body, infecting other tissues or organs.

In an attempt to rectify this, certain anticancer substances are sometimes used, in the context of chemotherapy, to combat the formation of metastases, but these chemical substances often produce undesirable side effects in patients, for example neuropathic pains.

The problem that arises is then to provide an effective composition or medicinal product for preventing, limiting, slowing or stopping tumor cell migration so as to limit or prevent the formation of metastases in an individual with cancer, in particular a composition or a medicinal product capable of killing or eradicating some or all of the free cancer cells carried by the blood of an individual, typically a human patient, notably the cancer cells released during surgery with the aim of removing a cancerous site from said individual, and without producing side effects in the latter.

### SUMMARY

The solution according to the invention is a gas composition containing xenon as active principle for use for preventing or for limiting tumor cell migration and/or limiting or preventing the formation of metastases, in an individual with cancer.

The following definitions are used in the context of the invention:
“patient” means an individual with cancer, whether a man, a woman, a child, a neonate, etc.;
“free cancer cell” means a cell not attached to a cancerous tissue or organ (i.e. a cancerous site) but derived from said cancerous site, and that can be transported;
“%” means percentage by volume, i.e. vol %, unless stated otherwise;
the terms “prevent”, “limit”, “slow” or “stop” are considered as roughly equivalent, basically to denote “prevent” or “combat”.

As appropriate, the gas composition of the invention used as inhalable medicinal product may comprise one or more of the following technical characteristics:
it can be administered by inhalation, for example by means of a breathing mask;
it can be administered by insufflation, for example by means of an endoscope, a tube suitable for this use or similar;
prevention or limitation of metastasis formation is obtained by the action of xenon on said free cancer cells;
xenon acts on the free cancer cells carried by the blood;
xenon acts on the free cancer cells by blocking the metastatic action of at least a proportion of said free cancer cells;
xenon is administered while the patient is undergoing surgery for removing the cancerous site from said patient;
administration of xenon begins before during surgery for the purpose of removing the cancerous site from said patient, is continued during surgery, preferably the administration of xenon is continued after the procedure;
xenon acts on the free cancer cells carried by the blood that are released during surgery for the purpose of removing a cancerous site from said patient;
the patient has a so-called “solid” cancer selected from liver, kidney, brain, skin, breast or colorectal cancer;
the effective proportion of xenon by volume is between 5 and 79%;
the effective proportion of xenon by volume is between 5 and 75%;
the effective proportion of xenon by volume is between 5 and 70%;
the proportion of xenon by volume is at least 10%;
the proportion of xenon by volume is at least 20%;
the proportion of xenon by volume is at least 30%;
the proportion of xenon by volume is at least 40%;
the proportion of xenon by volume is at least 50%;
the proportion of xenon by volume is at least 60%;
the proportion of xenon by volume is at least 65%;
the proportion of xenon by volume is between 62 and 75%;
the proportion of xenon by volume is between 65 and 73%;
it additionally contains a proportion of oxygen (O₂) by volume of at least 21%, typically between 21% and 50%, in particular between 21 and 35% of oxygen;
it is administered by inhalation for at least 15 minutes per day, preferably at least 30 min/day, more preferably at least 1 h/day;
it is administered by inhalation for a sufficient time to obtain an observable effect in the day or days following its administration by inhalation;
it is administered by inhalation continuously or fractionally, i.e. in several repetitions or several times;
optionally, it is co-administered with a substance for anticancer chemotherapy, i.e. it is administered before, during and/or after administration of the substance for anticancer chemotherapy;
it is administered by inhalation during at least the whole period of time of treatment of the patient with the substance for anticancer chemotherapy. Thus, if the patient has to take the substance for anticancer chemotherapy for 6 months for example, then the medicinal product is also administered for this same duration of 6 months, or even for a bit longer to mitigate any delayed effects;
it further contains an additional gas selected from argon, helium, neon, krypton, NO, CO, hydrogen sulfide (H₂S) and nitrogen, including from the air;
the patient is a human being, i.e. a man or a woman, including children, adolescents or any other group of individuals, for example neonates or the elderly;
it is ready to use;
it is packaged in a gas bottle, in particular a gas bottle or cylinder equipped with a tap or a tap with an integral pressure reducing valve. Preferably, the tap or the tap with an integral pressure reducing valve is protected by a protective cowling.

In other words, according to the present invention, it is proposed to administer, by inhalation or insufflation, a composition based on xenon, preferably containing more than 60% xenon and the remainder oxygen, in order to prevent or limit the of the xenon as active principle for use for preventing or for limiting tumor cell migration so as to limit or prevent metastasis formation in an individual with cancer, notably with the aim of killing some or all of the cancer cells that may have been released during a surgical operation that aimed to remove a cancerous site.

The concentration and/or the duration of administration that is the most suitable for a given patient may be selected empirically by the treating personnel, for example as a function of the patient's state of health or physical condition, the severity of the pain, their sex, their age, the type of cancer, type of anticancer agent co-administered, etc.

The xenon-based gas according to the invention can be used in the context of a method of therapeutic treatment, in which the gas mixture is administered, for example by inhalation by means of a breathing mask either connected directly to a source of xenon at the required concentration, for example a ready-to-use gas bottle, or to the outlet of a gas mixer supplied by several sources of gas (O₂, Xe, etc.) so as to obtain the desired mixture; or connected to a respiratory ventilator supplied with the desired gas or gases.

In other words, the present invention is therefore based on the use of a therapeutic gas mixture containing xenon as active product in an effective proportion for making an inhalable medicinal product with therapeutic effects intended to limit tumor cell migration, namely free cancer cells carried by the blood of a cancer patient who has undergone surgery with the aim of removing the tissues or the organ affected by said cancer.

### BRIEF DESCRIPTION OF THE DRAWINGS

The present invention will now be better understood owing to the following description and the examples hereunder, which are given purely for illustration, and referring to the appended figures, where:

FIG. 1 shows the migration of ER− cancer cells of type MDA-MB-231 exposed or not exposed to an atmosphere containing gaseous xenon.

FIG. 2 shows the migration of ER+ cancer cells of type MCF7 exposed or not exposed to an atmosphere containing gaseous xenon.

### DESCRIPTION OF PREFERRED EMBODIMENTS

To demonstrate the efficacy of the xenon-based gas according to the invention for combating the migration of free cancer cells carried by the blood of a cancer patient, tests were carried out in the following conditions.

ER− and ER+ cancer cells (Estrogen Receptor) of a human adenocarcinoma (breast cancer), namely lines of MDA-MB-231 and MCF7 cells, were deposited on plates coated with collagen, where they adhered. These cell lines are available commercially.

The plates with the adhering cells were then placed in a sealed chamber, saturated, for 1 h, 3 h and 5 h, with:
either a control atmosphere (control), namely a gas mixture formed from 25% O₂, 5% CO₂ and 70% N₂ (vol %);
or a test atmosphere based on xenon according to the invention, namely a gas mixture formed from 25% O₂, 5% CO₂ and 70% xenon (vol %).

Cell survival was determined after 24 hours, using an MTT (for methylthaizolyldiphenyl-tetrazolium bromide) test. This test is a conventional test for determining cellular viability. It is based on detection of living cells only, via the activity of their mitochondrial dehydrogenases, which makes it possible to evaluate cytotoxicity, proliferation and/or cellular activation.

For its part, cell migration at 24 h was determined after 24 hours, using fluorescence-labeled cells in the context of an Oris™ cell migration test. In this test, the labeled cells migrate into a target zone, where they are then quantified by a fluorescent plate reader for measuring the relative fluorescent units (RFU)).

These tests showed that xenon does not have a significant effect on cell survival.

In contrast, xenon inhibits cell migration both for MDA-MB-231 cells and for MCF7 cells relative to the control group (cells not brought into contact with xenon), in the proportions shown in the following table, regardless of the time of exposure to the gas.

| | TABLE | | |
|---|---|---|---|
| | % cell migration relative to the control | | |
| | Time of exposure to | | |
| | the xenon (hours) | | |
| | 1 h | 3 h | 5 h |
| MDA-MB-231 | 59 ± 13% | 64 ± 10% | 71 ± 9% |
| cells | (n = 4, P = 0.02) | (n = 4; P = 0.01) | n = 3, P = 0.04 |
| MCF7 cells | 58 ± 12% | 65 ± 12% | 65 ± 12% |
| | n = 4, P = 0.01 | n = 4, P = 0.03 | n = 3, P = 0.04 |

n: number of assays/tests
P = probability value (significance demonstrated if p < 0.05)

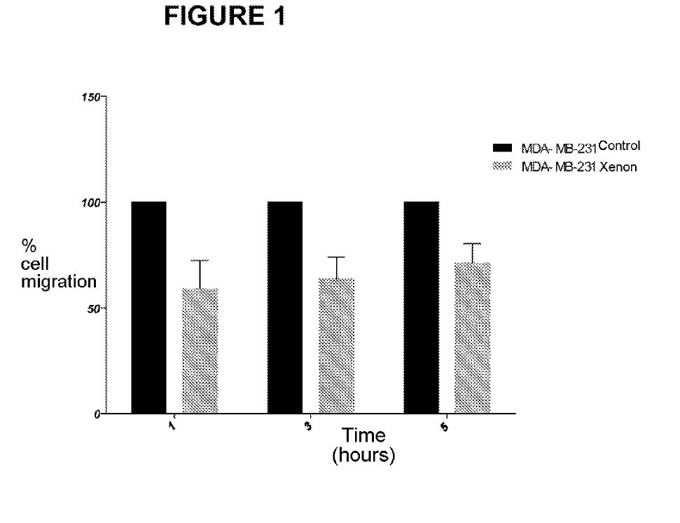

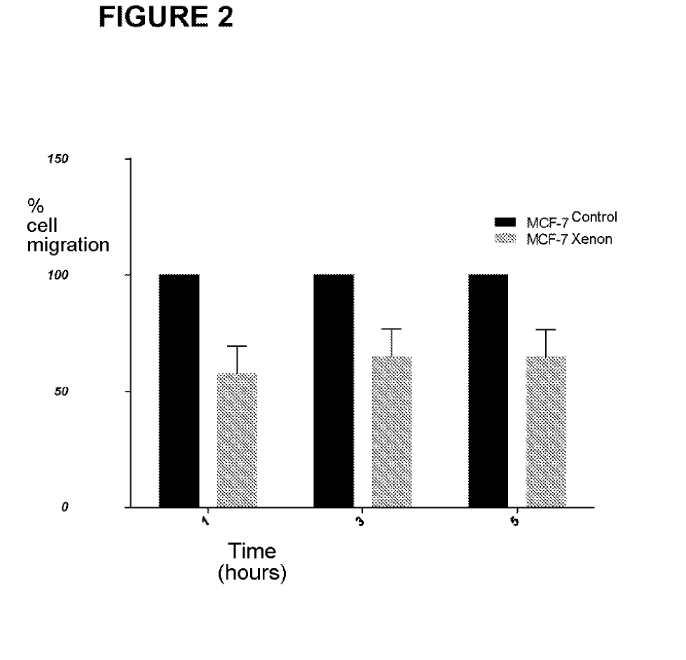

These results are illustrated in FIGS. 1 and 2.

More precisely, as already noted, several tests (3 or 4 tests) were conducted with the different gas atmospheres for different exposure times, and the results were expressed in the form of relative fluorescent units (RFU).

The migration of the cancer cells exposed to the atmosphere containing xenon was calculated as a percentage (%) relative to that of the cells exposed to the control atmosphere, i.e. exposed to an atmosphere without xenon (RFU fixed at 100%), and in the same conditions.

A two-way t-test for statistical analysis of the difference between the cell lines and the probability values P below 0.05 were considered significant.

As can be seen from FIG. 1, xenon (Xe) significantly reduces the migration of ER- MDA-MB-231 cells, as well as that of ER+MCF7 cells, as illustrated in FIG. 2, starting from 1 hour of exposure.

This demonstrates the efficacy of gaseous xenon for preventing or for limiting tumor cell migration so as to limit or prevent metastasis formation in an individual with cancer.

1-15. (canceled)

16. A method of preventing or limiting tumor cell migration and/or limiting or preventing metastasis formation in an individual with cancer, comprising a step of exposing the tumor cell to a gas composition containing xenon as active agent, resulting in the prevention or limitation of the tumor cell migration.

17. The method of claim 16, wherein the prevention or limitation of tumor cell migration is obtained by the action of xenon on a free cancer cell.

18. The method of claim 17, wherein the xenon of the gas composition acts on the free cancer cell carried by the blood.

19. The method of claim 16, wherein the xenon of the gas composition acts on a plurality of free cancer cells by blocking the metastatic action of at least a proportion of said plurality of free cancer cells

20. The method of claim 16, wherein an effective proportion of xenon by volume in the gas composition is between 5 and 79%.

21. The method of claim 20, wherein the effective proportion of xenon by volume is at least 50%.

22. The composition method of claim 16, wherein the gas composition additionally contains a proportion of oxygen by volume (O₂) of at least 21%.

23. The method of claim 16, wherein the gas composition further contains an additional gas selected from air, argon, helium, neon, krypton, NO, CO, hydrogen sulfide (H₂S), N₂O and nitrogen.

24. The method of claim 16, wherein t the gas composition is co-administered with a substance for anticancer chemotherapy.

25. The composition method of claim 16, wherein the individual is a human being.

26. The method of claim 16, wherein the gas composition is administered by inhalation or by insufflation.

27. The method of claim 16, wherein the individual is a patient that has liver, kidney, brain, skin, breast or colorectal cancer.

28. The method of claim 16, wherein the gas composition is administered while the individual undergoes surgery for the purpose of removing a cancerous site in said individual.

29. The method of claim 16, wherein the administration of the gas composition begins before or during a surgery for a purpose of removing a cancerous site in said individual, and is continued during the surgery.

30. The method of claim 16, wherein the xenon of the gas composition acts on a plurality of free cancer cells carried by a blood that are released during a surgery performed for a purpose of removing a cancerous site in said individual.
